(19) United States
(12) Patent Application Publication
(10) Pub. No.: US 2026/0007835 A1
(43) Pub. Date: Jan. 8, 2026

(54) MULTI-BEVEL INSERTION NEEDLE WITH OPEN CHANNEL FOR MEDICAL APPLICATIONS

(71) Applicant: AITA BIO INC., New York, NY (US)

(72) Inventors: Brishelle Aquise, West New York, NJ (US); Russell Cole, River Vale, NJ (US)

(21) Appl. No.: 19/117,055

(22) PCT Filed: Oct. 28, 2023
(86) PCT No.: PCT/US23/36168

§ 371 Date: Mar. 30, 2025

Related U.S. Application Data
(60) Provisional application No. 63/420,821, filed on Oct. 31, 2022.

(51) Int. Cl. A61M 5/32

(52) U.S. Cl. CPC A61M 5/3286; A61M 2005/3201

(57) ABSTRACT

A device for delivering medication to a user. The device includes an insertion needle for penetrating tissue of the user and deploying an infusion catheter and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle comprising: a channel along a length thereof for carrying and deploying the infusion catheter and/or CGM sensor into the user: first and second ledges that define a first opening to the channel that extends along a length of the insertion needle, the first and second ledges define a ledge plane: a first bevel surface toward a distal end of the insertion needle, the first bevel surface extends from the first and second ledges, the first bevel surface defines a second opening to the channel that enables the infusion catheter and/or CGM sensor to be deployed through the second opening: and a second bevel surface at a distal end of the insertion needle adjacent the first bevel surface, the second bevel surface configured as a tip for penetrating the tissue of the user.

### CROSS REFERENCE TO RELATED APPLICATIONS

This application claims priority to U.S. provisional application No. 63/420,821 filed Oct. 31, 2022, entitled “Multi-Bevel Insertion Needle With Open Channel” which is incorporated by reference herein.

### FIELD OF THE INVENTION

The present invention relates to a multi-bevel insertion needle with an open channel for delivering an infusion catheter and/or a CGM sensor.

### BACKGROUND OF THE INVENTION

Insulin pumps help people with diabetes to conveniently manage their blood sugar. These devices deliver insulin at specific times. Insulin patch pumps or pods are one type of insulin pump. The pumps are wearable devices that adhere to the skin of a user using an adhesive patch. The pumps deliver insulin from a chamber and infusion catheter based on continuous glucose monitoring (CGM) sensor readings that are acquired by a separate device. These devices are typically bulky, cumbersome and, often times, painful to apply and wear.

It would be advantageous to provide improvements to insulin pumps and diabetes management described above.

### SUMMARY OF THE INVENTION

A multi-bevel insertion needle with an open channel for a device for delivering medication is disclosed.

In accordance with an embodiment of the present disclosure, a device for delivering medication to a user, the device including an insertion needle for penetrating tissue of the user and deploying an infusion catheter for delivering the medication and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle comprising: a channel along a length thereof for carrying and deploying the infusion catheter and/or CGM sensor into the user; first and second ledges that define a first opening to the channel that extends along a length of the insertion needle, the first and second ledges define a ledge plane; a first bevel surface toward a distal end of the insertion needle, the first bevel surface extends from the first and second ledges, the first bevel surface defines a second opening to the channel that enables the infusion catheter and/or CGM sensor to be deployed through the second opening; and a second bevel surface at a distal end of the insertion needle adjacent the first bevel surface, the second bevel surface configured as a tip for penetrating the tissue of the user.

In accordance with another embodiment of the present disclosure, an insertion needle for penetrating tissue of the user and deploying an infusion catheter for infusing medication into a user and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle comprising: a channel along a length thereof for carrying and deploying the infusion catheter and/or CGM sensor into the user; first and second ledges that define a first opening to the channel that extends along a length of the insertion needle, the first and second ledges define a ledge plane; a first bevel surface toward a distal end of the insertion needle, the first bevel surface extends from the first and second ledges, the first bevel surface defines a second opening to the channel that enables the infusion catheter and/or CGM sensor to be deployed through the second opening; and a second bevel surface at a distal end of the insertion needle adjacent the first bevel surface, the second bevel surface configured as a tip for penetrating the tissue of the user.

In accordance with another embodiment of the present disclosure, an insertion needle for penetrating tissue of the user and deploying an infusion catheter for infusing medication into a user and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle including a tube comprising: a channel along a length thereof for carrying and deploying the infusion catheter and/or CGM sensor into the user; first and second ledges that define a ledge plane and a first opening to the channel that extends along the length of the tube; a first bevel surface toward a distal end of the tube defines a second opening to the channel that enables the infusion catheter and/or CGM sensor to be deployed through this second opening; and a second bevel surface at a distal end of the tube adjacent the first bevel surface, the second bevel surface configured as a tip for penetrating the tissue of the user.

In accordance with yet another embodiment of the present disclosure, a device for delivering insulin to a user, the device including an micropump for pumping the insulin and an insertion needle for penetrating tissue of the user and deploying an infusion catheter for delivering the insulin and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle comprising: a channel along a length thereof for carrying and deploying the infusion catheter and/or CGM sensor into the user; first and second ledges that define a first opening to the channel that extends along a length of the insertion needle, the first and second ledges define a ledge plane; a first bevel surface toward a distal end of the insertion needle, the first bevel surface extends from the first and second ledges, the first bevel surface defines a second opening to the channel that enables the infusion catheter and/or CGM sensor to be deployed through the second opening; and a second bevel surface at a distal end of the insertion needle adjacent the first bevel surface, the second bevel surface configured as a tip for penetrating the tissue of the user.

In accordance with another embodiment of the disclosure, an insertion needle for penetrating tissue of the user and deploying an infusion catheter for infusing insulin into a user and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle including a tube comprising: a channel along a length thereof for carrying and deploying the infusion catheter and/or CGM sensor into the user, first and second ledges that define a ledge plane and a first opening to the channel that extends along the length of the tube; a first bevel surface toward a distal end of the tube defines a second opening to the channel that enables the infusion catheter and/or CGM sensor to be deployed through this second opening; and a second bevel surface at a distal end of the tube adjacent the first bevel surface, the second bevel surface configured as a tip for penetrating the tissue of the user.

### BRIEF DESCRIPTION OF DRAWINGS

FIGS. 1 and 2 depict perspective and front views respectively of an example three bevel U-shaped insertion needle for a device for delivering medication.

FIGS. 3 and 4 depict front and perspective views respectively of an example two bevel U-shaped insertion needle for a device for delivering medication.

FIGS. 5-7 depict views of another example two bevel square shaped insertion needle for a device for delivering medication.

FIGS. 8 and 9 depict views of another example three-bevel square shaped insertion needle for a device for delivering medication.

FIGS. 10-12 depict views of another example three-bevel square shaped insertion needle with a back bevel for a device for delivering medication.

FIGS. 13-17 depict views of the formation of a U-shaped insertion needle.

FIG. 18 depicts a block diagram of example components of the device for delivering insulin and needle cartridge assembly of the infusion system.

### DETAILED DESCRIPTION OF THE INVENTION

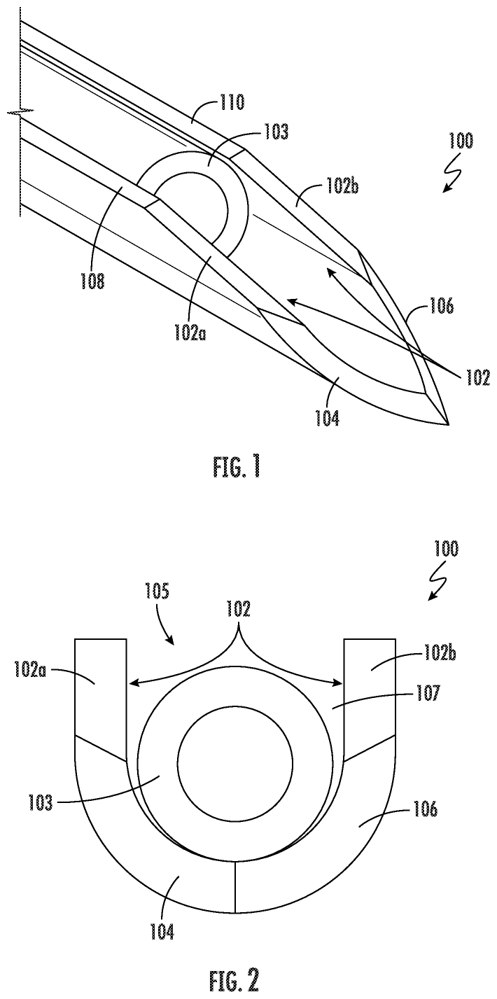

FIGS. 1 and 2 depict perspective and front views respectively of example three bevel U-shaped insertion needle 100 of a device (not shown) for delivering medication (delivery device) such as insulin (example). Insertion needle 100 is configured as (and may be referred to as) a carrier needle or introducer needle for carrying/introducing an infusion catheter (infusion catheter 103 below) used to deliver fluid to a user or a continuous glucose monitoring (CGM) sensor as described herein. Needle 100 is an example of a multi-bevel shaped needle. As disclosed herein, the multi-bevel needle may include two, three or more bevel surfaces.

Specifically, in this example, insertion needle 100 is a tube that is U-shaped (may be referred to as tube 100) with an open channel or lumen (i.e., opening to a channel) for receiving one or more components as described in more detail below. Insertion needle 100 is used for introducing and deploying these components (as described below) percutaneously into the tissue of a user. Examples of such components include an infusion catheter (e.g., infusion catheter 103) and/or a continuous glucose monitoring (CGM) sensor (not shown) that tracks glucose levels in the user.

In short, with respect to insertion needle 100 and other example insertion needles described hereinbelow, it is the shape of the needle tube (including width of sheet and open channel) and the geometry of a two or more bevel configuration toward the end of, i.e. at the tip of needle 100 (and others described hereinbelow) that decreases penetration forces and minimizes footprint on the user's tissue during insertion, thereby reducing the discomfort or pain induced due to the invasive nature of needle insertion. Needle 100 is described in more detail below.

Delivery Device. The delivery device (not shown) is part of an infusion system for diabetes management. The device is a wearable apparatus, system or pod in which continuous glucose monitoring (CGM), insulin delivery and control functionality are provided to ensure insulin is delivered at very precise rates and has the capability of detecting occlusions in real time. In one example, the device is applied by opening a sterile packaging, filling the reservoir with insulin, priming the fluid path, removing the adhesive backing, sticking the device to the desired body location, pushing a button assembly, removing and disposing a detachable activation mechanism (not described in detail herein).

The device incorporates, among other elements (as described below), a micropump or other pump as known to those skilled in the art that can be used for pumping fluid, valves used for regulating flow, actuators used for moving or controlling the micropump and valves and/or sensors used for sensing pressure and/or flow. The micropump may be used to infuse the insulin or other fluidic medication to the user (patient). Medication may include small molecule pharmaceutical solutions, large molecule or protein drug solutions, saline solutions, blood or other fluids known to those skilled in the art. Insulin is an example of medication or fluid that is described in this application. However, micropump may be used in other environments known to those skilled in the art.

The device also includes reservoir, a microcontroller unit (MCU) (not shown), insertion needle 100, infusion catheter and/or a CGM sensor and a battery and power controller (not shown). The reservoir is configured to receive and store insulin for its delivery over a course of about three days, or as needed. However, reservoir size may be configured for storing any quantity of fluid as required. The micropump fluidly communicates with reservoir to enable infusion as needed. CGM, as known to those skilled in the art, tracks patient glucose levels and permits those levels to be used in algorithms that control flow rate. MCU controls the operation of the micropump to deliver insulin through the infusion catheter from the reservoir at specific doses, i.e., flow rates over specified time intervals, based on CGM data converted to desired flow rate via control algorithms. The battery and power controller controls the power to the MCU and the micropump to enable those components to function properly as known to those skilled in the art. The CGM is powered by battery and the power controller through the MCU. (An example device (device 1800) and its components is shown in FIG. 18.)

Insertion needle 100. In the example shown in FIGS. 1 and 2, insertion needle 100 may be part of and deployed by, for example, a needle cartridge assembly or other driving mechanism that is used to insert or introduce insertion needle 100 percutaneously in the tissue (layer) of a user (patient). As described above, insertion needle 100 is a tube that is U-shaped (may also be referred to as tube 100) as shown with a rounded base. The U-shaped tube 100 has an opening 105 or open side that extends through the length of the tube that leads to channel 107 (open channel) for storing a component such as an infusion catheter 103 and/or a CGM sensor (CGM sensor not shown). However, in other examples, the tube may be partially open sided or may even define an open window. Insertion needle 100 is preferably constructed of steel but it may be any rigid material known to those skilled in the art.

Further, in this example, there are three bevel surfaces at the distal end of the tube (insertion needle 100), one of which is primary bevel surface 102. Bevel surface 102 comprises two sections 102a, 102b that appear on both sides of the U-shaped tube and together defines a bevel plane for bevel surface 102. Sections 102a, 102b slope toward the tip of needle 100. Bevel surface 102 extend from ledges 108,110 and functions as a middle surface or transitional wall between ledges 108, 110 of the tube and bevel surfaces 104, 106. Ledges 108, 110 together not only defines a ledge plane, but also defines the opening that leads to the channel (open channel) for receiving infusion catheter 103 and/or CGM sensor (for example). Two bevel surfaces 104, 106 are configured as a tip used for percutaneously penetrating tissue of the user. Specifically, bevel surfaces 104, 106 are adjacent bevel surface 102 and define planes respectively that intersect in a triangular or apex configuration, creating the tip used to penetrate the tissue of the user. The planes of bevel surfaces 102, 104, 106 are each different in angle from one another. Bevel surfaces 104, 106 slope both outwardly with respect to one another and slope toward the distal end of insertion needle 100. A bevel, bevel edge or bevel surface is defined as sloping surface from a horizontal or vertical surface or wall. Two or more surfaces, on the sides of the U-shaped insertion needle 100 such as that shown in FIGS. 1 and 2, having the same sloping surface or that are configured in the same plane, are considered or constitute the same bevel surface.

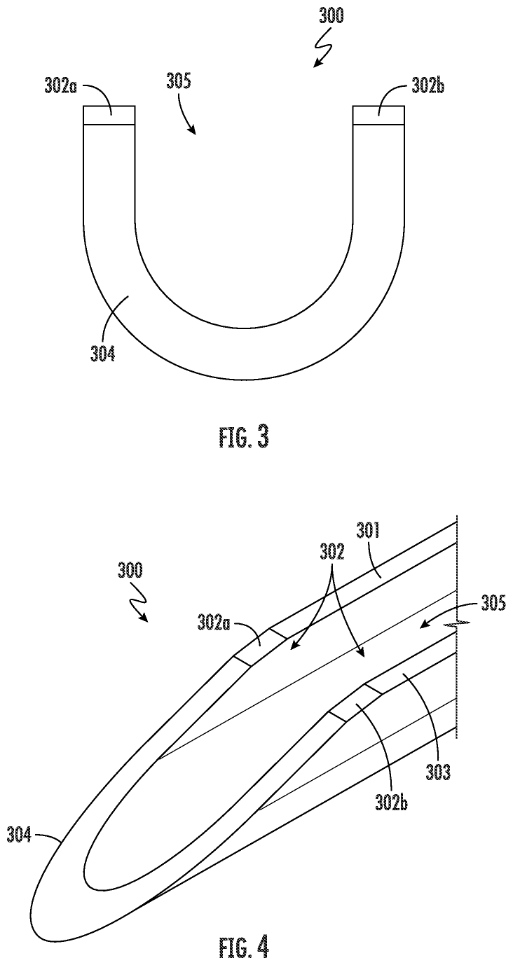

Insertion needle 300. FIGS. 3 and 4 depict front and perspective views respectively of another example U-shaped insertion needle 300 for a device for delivering medication, but needle 300 has two bevels. (In this example, needle 300 does not depict (carry) an infusion catheter and/or CGM sensor for illustration purposes.) This shape is similar to the example described above. In this example, bevel surface 302 comprises two sloped sections 302a, 302b that appear on both sides of the U-shaped tube that defines an opening into a channel for storing the infusion catheter and/or CGM sensor. However, this example differs to that in FIGS. 1 and 2 above because bevel surface 304 is defined in a singular surface in one plane and it is configured as the penetration tip. Similar to the example above, bevel surface 302 extends between ledges 301,302 of the needle 300 (tube) and bevel surface 304. Ledges 301,303 together define a ledge plane and also define the opening that leads to the channel 305 (open channel) for receiving an infusion catheter and/or CGM sensor (for example).

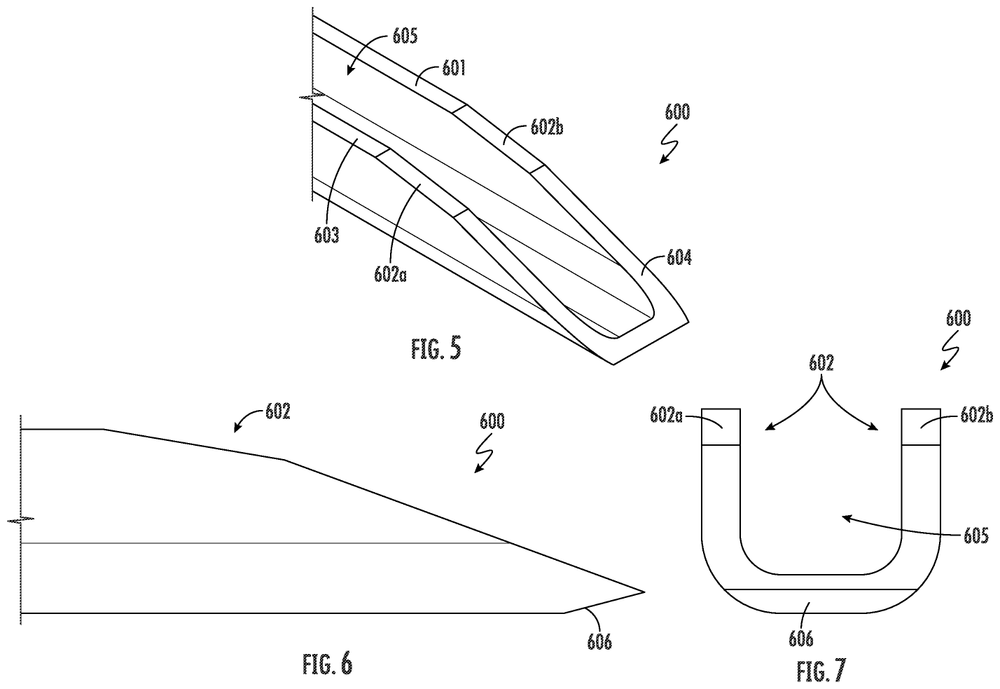

Insertion needle 600. FIGS. 5, 6 and 7 depict views of another example three bevel square shaped insertion needle 600 for a device for delivering medication. (In this example, needle 600 does not depict (carry) an infusion catheter and/or CGM sensor for illustration purposes.) Similar to the examples above, the insertion 600 is opened sided that leads into a channel within needle 600. In this example, there are two top adjacent bevel surfaces 602, 604 and bevel surface 606 on the bottom of needle 600. Bevel surfaces 604,606 define a tip for tissue penetration. Similar to the examples above, bevel surface 602 (primary) comprises two sloped sections 602a, 602b that appear on both sides of the square shaped tube that are positioned in the same plane. The square-shaped tube is also open ended, and the opening extends along the length of needle 600. However, the open may extend at different lengths along needle 600. Similar to the examples above, bevel surface 602 extends between ledges 601,603 of the needle 600 (tube) and bevel surface 604. Ledges 601,603 together define a ledge plane and also define the opening that leads to the channel 605 (open channel) for receiving an infusion catheter and/or CGM sensor (for example).

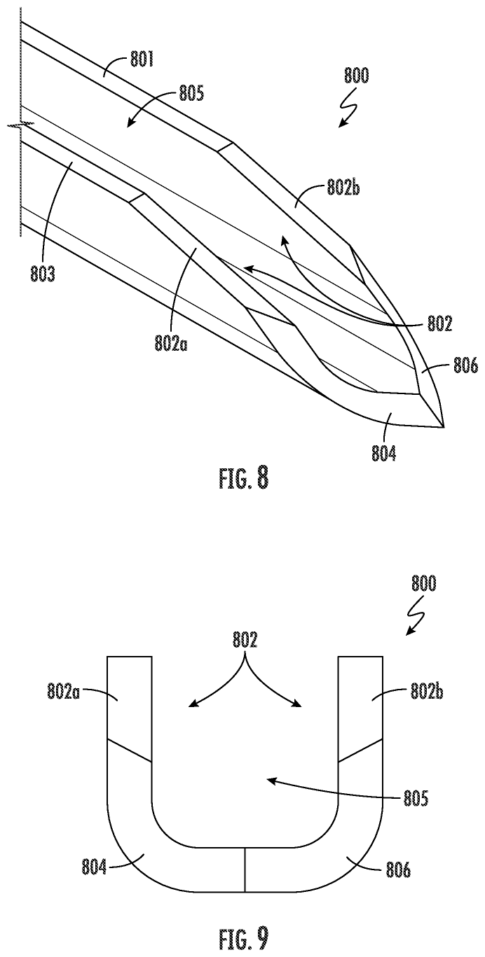

Insertion needle 800. FIGS. 8 and 9 depict views of another example three-bevel open square shaped insertion needle 800 for a device for delivering medication. (In this example, needle 300 does not depict (carry) an infusion catheter and/or CGM sensor for illustration purposes.) In this example, needle 800 includes primary bevel surface 802 and adjacent bevel surfaces 804,806 that together function as a tip for tissue penetration. Bevel surface 802 (primary) comprises two sloped sections 802a, 802b that appear on both sides of the square-shaped tube that are positioned in the same plane. Ledges 801, 803 of the tube 800 together not only define a ledge plane, but also define the opening that leads to the channel 805 (open channel) for receiving an infusion catheter and/or CGM sensor (for example). Bevel surface 802 extends from ledges 801,803 and between ledges 801,803 and bevel surfaces 804,806. Bevel surfaces 804,806 are configured to slope outwardly with respect to bevel surface 802 and each bevel surface 804,806 are flat (i.e., each in its own plane). (However, in other embodiments, bevel surfaces 804,806 may vary in slope defining various planes, i.e., curved toward the tip of the needle.) The square-shaped needle 800 is also open ended and the opening extends along the length of needle 800 that defines a channel within needle 800. However, the opening may extend at different lengths along needle 800.

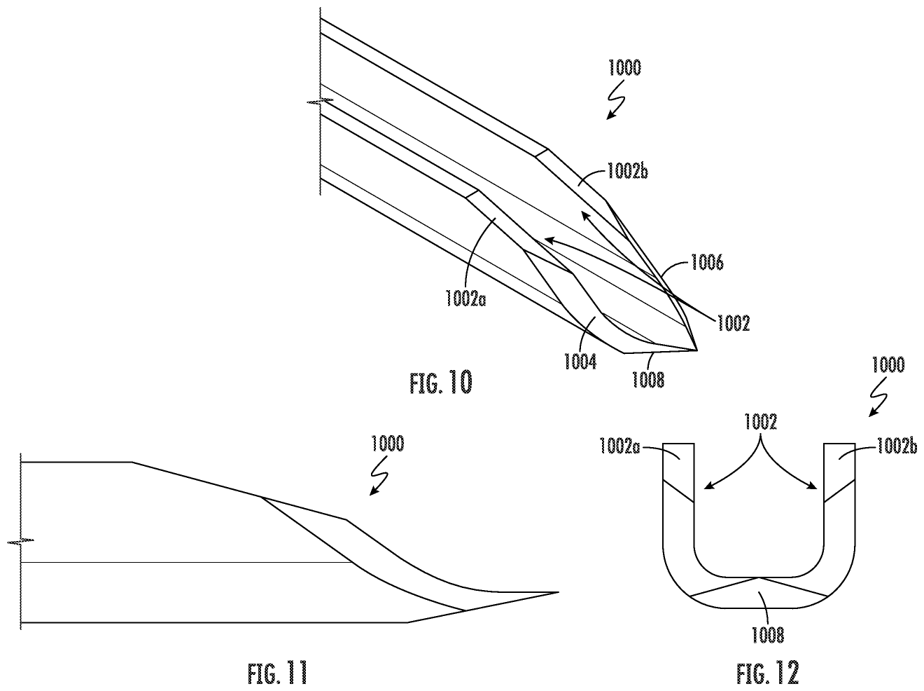

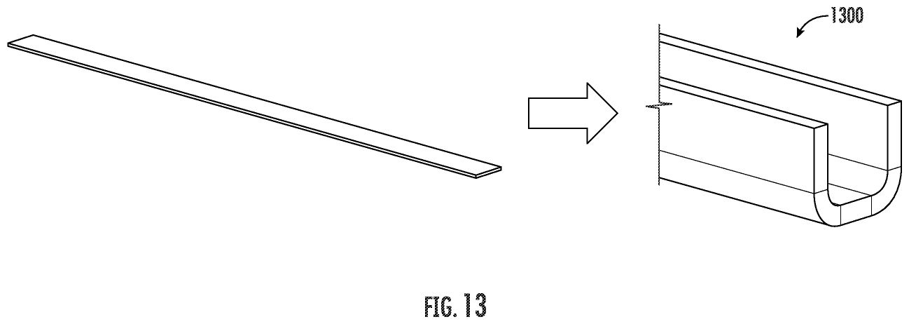

Insertion needle 1000. FIGS. 10, 11 and 12 depict views of another example three-bevel open square shaped insertion needle 1000 with a back bevel for a device for delivering medication. (In this example, needle 1000 does not depict (carry) an infusion catheter and/or CGM sensor for illustration purposes.) In this example, needle 1000 includes primary bevel surface 1002, adjacent bevel surfaces 1004, 1006 and bottom bevel surface 1008. Bevel surfaces 1004, 1006 and bottom bevel surface 1008 that together function as a tip for tissue penetration. Bevel surfaces 1004, 1006 are flat (each its own plane) and slope outwardly with respect to bevel surface 1002. Bevel surface 1002 (primary) comprises two sloped sections 1002a, 1002b that appear on both sides of the square-shaped tube that are positioned in the same plane. The square-shaped needle 1000 is also open sided in that the opening extends along the length of needle 1000 that defines a channel within needle 1000. However, the opening may extend at different lengths along needle 1000.

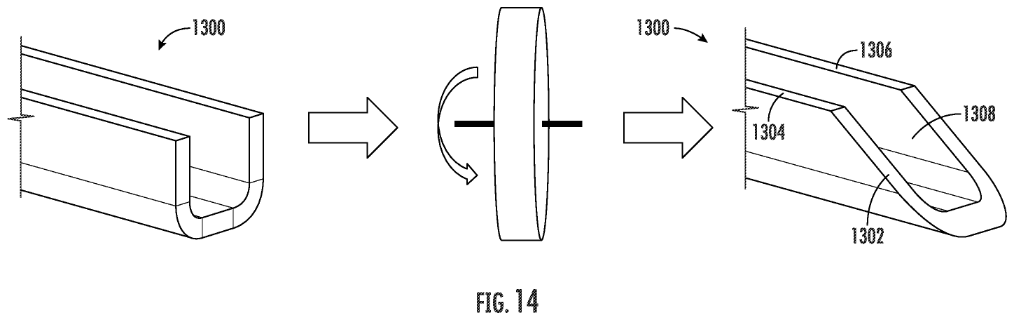

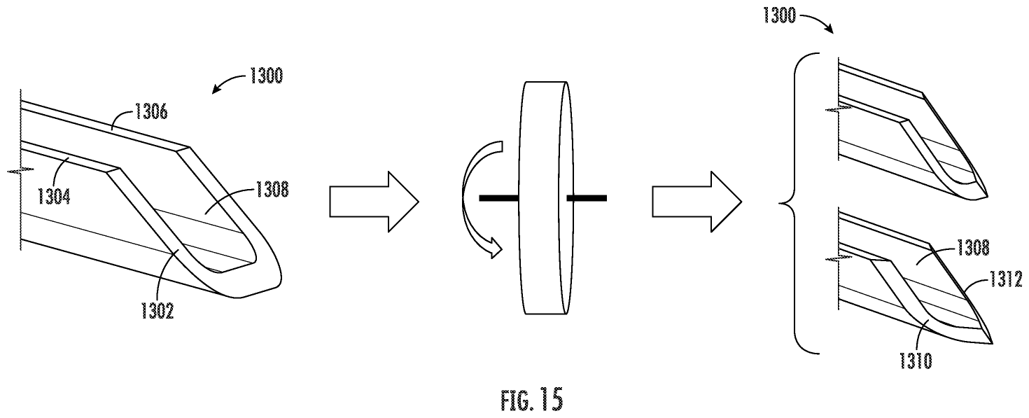

Formation. FIGS. 13, 14, 15, 16 and 17 depict views of the formation of a U-shaped insertion needle. In FIG. 13, a U-shaped tube 1300 is formed from a thin metal sheet as shown. The width of the sheet (selection) dictates opening in tube 1300 to access the channel within tube 1300. In FIG. 14, tube 1300 is grinded to form a bevel surface 1302 on the end of the tube from the ledges 1304, 1306 that define the opening and channel 1308 of the tube. The grinding process proceeds where bevel surface 1302 is grinded down to form two-sided bevel surfaces 1310, 1312 that extend outwardly as shown in FIG. 15.

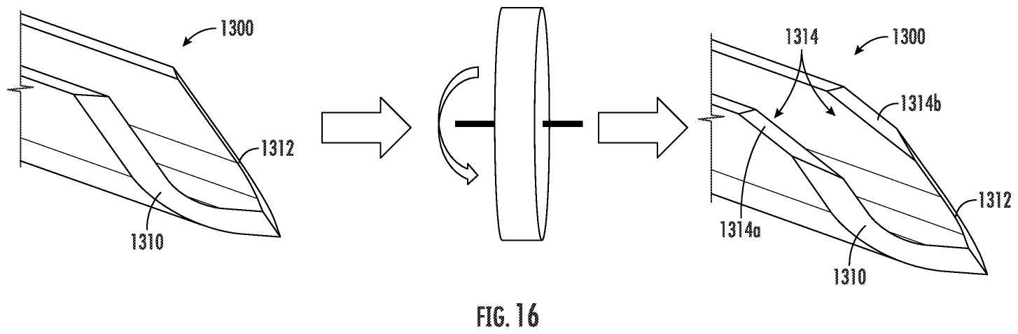

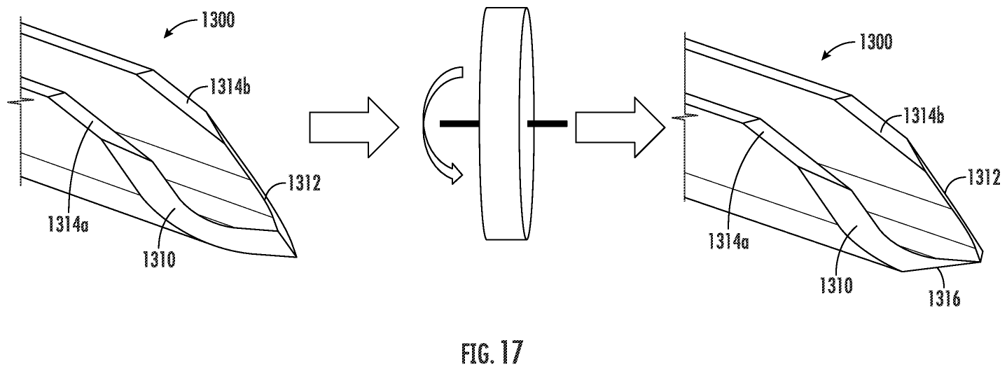

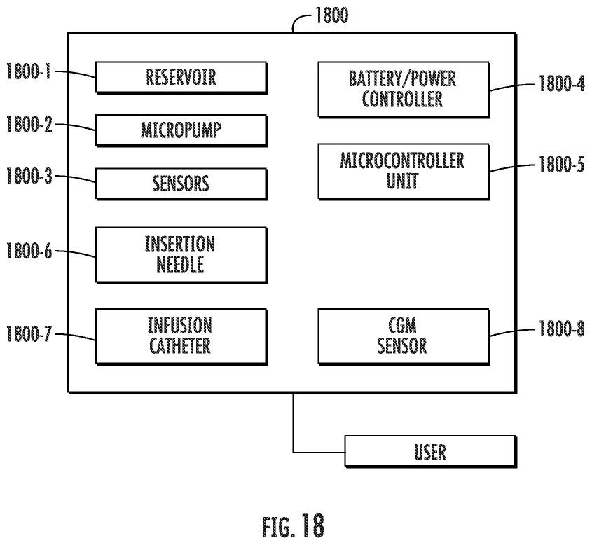

In FIG. 16, a primary bevel surface 1314 (1314a, 1314b) is formed by grinding down the top of both bevel surfaces 1310,1312. The resulting primary bevel surface 1314 functions as a transition surface between ledges 1304, 1306 of the tube 1300 toward the two side bevels 1310, 1312. In FIG. 17, 1300 tube is grinded on its bottom to form bevel surface 1316. The two bevel surfaces 1310, 1312 and bottom bevel surface 1316 together function as a tip for penetrating user tissue. This formation process may be also employed for creating the other insertion needles described herein.

The example of the geometry of the needles described hereinabove require less penetration forces, thereby reducing the discomfort or pain induced due to the invasive nature of the insertion.

FIG. 18 depicts a block diagram of example components of (1) device 1800 for delivering insulin that incorporates (or used with) the insertion needles described herein. Specifically, device 1800 incorporates several components or modules (not shown) in the fluidic pathway including reservoir 1800-1 for storing the insulin, micropump 1800-2 for pumping the insulin, sensors 1800-3 (e.g., pressure) for sensing various parameters in the system and user and tubing connecting infusion needle 1802-1 to reservoir 1800-1 within cartridge assembly 1802. Device 1800 also includes microcontroller unit (MCU) 1800-4 and battery and power controller 1800-5. Device 1800 further incorporates insertion needle 1800-6 (described herein) along with infusion catheter 1800-7 and CGM sensor 1800-8. CGM, as known to those skilled in the art, tracks user glucose levels and permits those levels to be used in algorithms that control flow rate. MCU 1800-4 controls the operation of micropump 1800-2. Integrated infusion catheter 1802-1 and CGM sensor 1802-2 subassembly are shown as separate components in FIG. 18 for illustration purposes, but they are together as they are introduced as described hereinabove.

Alternatively, a driving mechanism may be used to drive insertion needle 1800-6 along with infusion catheter 1800-7 into a tissue layer of a user. The mechanism may be a cartridge assembly as described above. The mechanism may be part of device 1800 or a separate component such as a cartridge assembly and the cartridge assembly may incorporate insertion needle 1800-6 and infusion catheter 1800-7. CGM sensor 1800-8 may also be incorporated into the cartridge assembly or other driving mechanism.

Reservoir 1800-1 is configured to receive and store insulin for its delivery over a course of about three days, or as needed. However, reservoir size may be configured for storing any quantity of fluid as required.

MCU 1800-5 electronically communicates with sensors 1800-3 and micropump 1800-2 as well as the CGM sensor 1802-8, as the monitoring components. Among several functions, MCU 1800-5 operates to control the operation of micropump 1800-2 to deliver insulin through insulin needle 1802-1 from reservoir 1800-1 at specific doses, i.e., flow rates over specified time intervals, based on CGM data converted to desired flow rate via control algorithms.

Battery and power controller 1800-4 controls the power to MCU 1800-5 and micropump 1800-2 to enable those components to function properly as known to those skilled in the art. CGM sensor 1800-8 is powered by battery and power controller 1800-4 through MCU 1800-5.

The components of device 1800 shown in FIG. 18 are only a few components. Those skilled in the art know that device 1800 may include additional components.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. A device for delivering medication to a user, the device including an insertion needle for penetrating tissue of the user and deploying an infusion catheter for delivering the medication and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle comprising:
a channel along a length thereof for carrying and deploying the infusion catheter and/or CGM sensor into the user;
first and second ledges that define a first opening to the channel that extends along a length of the insertion needle, the first and second ledges define a ledge plane;
a first bevel surface toward a distal end of the insertion needle, the first bevel surface extends from the first and second ledges, the first bevel surface defines a second opening to the channel that enables the infusion catheter and/or CGM sensor to be deployed through the second opening; and
a second bevel surface at a distal end of the insertion needle adjacent the first bevel surface, the second bevel surface configured as a tip for penetrating the tissue of the user.

2. The device of claim 1 wherein the first bevel surface and second bevel surface are not in a same plane.

3. The device of claim 1 wherein the insertion needle having a third bevel surface adjacent the second bevel surface, the second and third bevel surfaces slope outwardly with respect to each other and together form the tip for penetrating the tissue of the user.

4. The device of claim 1 wherein the insertion needle is a U-shaped tube or square shaped tube.

5. The device of claim 1 wherein the first bevel surface comprise first and second sections that slope with respect to a plane defined by the first and second ledges.

6. The device of claim 1 wherein the insertion needle includes a third bevel surface on the distal end of the insertion needle opposing the second bevel surface, the second bevel surface and third bevel surface together define the tip for penetrating tissue of the issuer.

7. The device of claim 1 wherein the insertion needle includes a third bevel surface adjacent the first bevel surface, the second and third bevel surfaces slope outwardly with respect to each other and downwardly toward the distal end of the insertion needle and together form the tip for penetrating the tissue of the user.

8. The device of claim 1 wherein the first opening extends along a portion of the length of the insertion needle that is configured to penetrate the tissue of the user.

9. The device of claim 1 wherein the first opening extends along an entire length of the insertion needle that is configured to penetrate the tissue of the user.

10. The device of claim 1 wherein the medication is insulin.

11. An insertion needle for penetrating tissue of the user and deploying an infusion catheter for infusing medication into a user and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle comprising:
a channel along a length thereof for carrying and deploying the infusion catheter and/or CGM sensor into the user;
first and second ledges that define a first opening to the channel that extends along a length of the insertion needle, the first and second ledges define a ledge plane;
a first bevel surface toward a distal end of the insertion needle, the first bevel surface extends from the first and second ledges, the first bevel surface defines a second opening to the channel that enables the infusion catheter and/or CGM sensor to be deployed through the second opening; and
a second bevel surface at a distal end of the insertion needle adjacent the first bevel surface, the second bevel surface configured as a tip for penetrating the tissue of the user.

12. The insertion needle of claim 11 wherein the first bevel surface and second bevel surface are not in a same plane.

13. The insertion needle of claim 11 wherein the insertion needle having a third bevel surface adjacent the second bevel surface, the second and third bevel surfaces slope outwardly with respect to each other and together form the tip for penetrating the tissue of the user.

14. The insertion needle of claim 11 wherein the insertion needle is a U-shaped tube or square shaped tube.

15. The insertion needle of claim 11 wherein the first bevel surface comprise first and second sections that slope with respect to a plane defined by the first and second ledges.

16. The insertion needle of claim 11 wherein the insertion needle includes a third bevel surface on the distal end of the insertion needle opposing the second bevel surface, the second bevel surface and third bevel surface together define the tip for penetrating tissue of the user.

17. The insertion needle of claim 11 wherein the insertion needle includes a third bevel surface adjacent the first bevel surface, the second and third bevel surfaces slope outwardly with respect to each other and downwardly toward the distal end of the insertion needle and together form the tip for penetrating the tissue of the user.

18. The insertion needle of claim 11 wherein the first opening extends along a portion of the length of the insertion needle that is configured to penetrate the tissue of the user.

19. The insertion needle of claim 11 wherein the first opening extends along an entire length of the insertion needle and is configured to penetrate the tissue of the user.

20. The insertion needle of claim 11 wherein the medication is insulin.

21. An insertion needle for penetrating tissue of the user and deploying an infusion catheter for infusing medication into a user and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle including a tube comprising:
a channel along a length thereof for carrying and deploying the infusion catheter and/or CGM sensor into the user,
first and second ledges that define a ledge plane and a first opening to the channel that extends along the length of the tube;
a first bevel surface toward a distal end of the tube defines a second opening to the channel that enables the infusion catheter and/or CGM sensor to be deployed through this second opening; and
a second bevel surface at a distal end of the tube adjacent the first bevel surface, the second bevel surface configured as a tip for penetrating the tissue of the user.

22. The insertion needle of claim 21 wherein the first bevel surface and second bevel surface are not in a same plane.

23. The insertion needle of claim 21 wherein the insertion needle having a third bevel surface, the second bevel surface configured adjacent the first bevel surface, the second and third bevel surfaces slope outwardly with respect to each other and together form the tip for penetrating the tissue of the user.

24. The insertion needle of claim 21 wherein the tube is U-shaped or square shaped.

25. The insertion needle of claim 21 wherein the first bevel surface comprise first and second sections that slope with respect to a plane defined by the first and second ledges.

26. The insertion needle of claim 21 wherein the insertion needle includes a third bevel surface on the distal end of the tube opposing the second bevel surface, the second bevel surface and third bevel surface together define the tip for penetrating tissue of the issuer.

27. The insertion needle of claim 21 wherein the insertion needle includes a third bevel surface, the second bevel surface configured adjacent the first bevel surface, the second and third bevel surfaces that slope outwardly with respect to each other and together form the tip for penetrating the tissue of the user.

28. The insertion needle of claim 21 wherein the medication is insulin.

29. A device for delivering insulin to a user, the device including an micropump for pumping the insulin and an insertion needle for penetrating tissue of the user and deploying an infusion catheter for delivering the insulin and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle comprising:
a channel along a length thereof for carrying and deploying the infusion catheter and/or CGM sensor into the user;
first and second ledges that define a first opening to the channel that extends along a length of the insertion needle, the first and second ledges define a ledge plane;
a first bevel surface toward a distal end of the insertion needle, the first bevel surface extends from the first and second ledges, the first bevel surface defines a second opening to the channel that enables the infusion catheter and/or CGM sensor to be deployed through the second opening; and
a second bevel surface at a distal end of the insertion needle adjacent the first bevel surface, the second bevel surface configured as a tip for penetrating the tissue of the user.

30. The device of claim 29 wherein the first bevel surface and second bevel surface are not in a same plane.

31. An insertion needle for penetrating tissue of the user and deploying an infusion catheter for infusing insulin into a user and/or a CGM sensor into the user, the insertion needle having a shape and geometry that are configured to decrease penetration forces and minimizes footprint on the user's tissue during insertion, thereby reducing the discomfort or pain induced due to needle insertion, the insertion needle including a tube comprising:
a channel along a length thereof for carrying and deploying the infusion catheter and/or CGM sensor into the user,
first and second ledges that define a ledge plane and a first opening to the channel that extends along the length of the tube;
a first bevel surface toward a distal end of the tube defines a second opening to the channel that enables the infusion catheter and/or CGM sensor to be deployed through this second opening; and
a second bevel surface at a distal end of the tube adjacent the first bevel surface, the second bevel surface configured as a tip for penetrating the tissue of the user.
